(19) United States
(12) Patent Application Publication
(10) Pub. No.: US 2007/0015984 A1
(43) Pub. Date: Jan. 18, 2007

(54) Electrode for measuring biosignal

(75) Inventors: Hyung-sok Yeo, Yongin-si (KR); Youn-ho Kim, Hwaseong-si (KR); Kun-soo Shin, Seongnam-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/454,849

(22) Filed: Jun. 19, 2006

(30) Foreign Application Priority Data
Jun. 30, 2005 (KR) 10-2005-0058641

(51) Int. Cl. A61B 5/04

(52) U.S. Cl. 600/372; 600/395

(57) ABSTRACT

An electrode for measuring a biosignal. The electrode includes: a sheet member formed. of an insulating material; a conductive member formed of a conductive material on a first surface of the sheet member; and an electrolyte member formed on the sheet member, comprising a gel-type electrolyte having tackiness, and having a base part covering the conductive member and a plurality of protrusions protruding from a surface of the base part which is directed to the skin.

### CROSS-REFERENCE TO RELATED APPLICATION

This application claims the benefit of Korean Patent Application No. 10-2005-0058641, filed on Jun. 30, 2005, in the Korean Intellectual Property Office, the disclosure of which is incorporated herein by reference.

### BACKGROUND OF THE INVENTION

1. Field of the Invention

The present invention relates to an electrode for measuring a biosignal, and more particularly, to an electrode for measuring a biosignal having an improved structure in which an impedance change or the insertion of a motion artifact is minimized.

2. Description of Related Art

An electrocardiograph (ECG) test is a type of test that is performed by attaching electrodes to the body. An electrocardiogram is used to evaluate electrical activity generated by the heart at rest and when active. That is, the electrical activity generated by the heart at rest and when active is spread into the whole body from the heart and causes an electrical potential difference in two locations of the chest. This electrical potential difference can be detected and recorded by electrodes attached to the skin. Such ECG tests are used to check heart abnormalities and are basically used in the diagnosis of heart diseases such as angina pectoris, myocardial infarction, and arrhythmia.

In general, apparatuses for conducting ECG tests include electrodes for measuring biosignals in contact with the skin and signal analyzers for interpreting and processing biosignals measured by the electrodes. The electrodes and the signal analyzers can be connected in wired or wireless ways. The electrodes for measuring biosignals can be generally classified into dry electrodes or wet electrodes depending on the presence of an electrolyte on the surface attached to the skin. Dry electrodes are mainly applied to the chest using an elastic band. However, when the dry electrodes are applied to the chest, the chest is pressed so that they are not suitable for long-term application. On the other hand, wet electrodes are attached to the chest using tacky electrolytes.

FIG. 1 illustrates an example of a conventional wet electrode 10. Referring to FIG. 1, an electrolyte 11 is disposed on a surface of the wet electrode 10. The electrolyte 11 is a gel-type electrolyte, has tackiness, and is attached to the skin S. Before the body makes a motion, for example, when the body bends forward or straightens, the area of the electrolyte 11 that contacts the skin S is sufficient, as illustrated in the diagram on the left of FIG. 1. However, when the body makes a motion, the area of the electrolyte 11 that contacts the skin S can be reduced, as illustrated in the right of FIG. 1. As such, the electrolyte 11 may be separated from the skin S, and the impedance between the skin S and the electrolyte 11 can change. This change of impedance causes the insertion of a motion artifact in a signal measured by the wet electrode 10 so that the quality of the signal is degraded. Furthermore, if perspiration flows between the electrolyte 11 and the skin S when the electrolyte 11 is attached to the skin S for a long time, the tackiness of the electrolyte 11 may be degraded as time elapses. As such, the possibility that the electrolyte 11 is separated from the skin S is high, and the impedance between the electrolyte 11 and the skin S is changed so that the quality of the signal measured by the wet electrode 11 may be degraded.

### BRIEF SUMMARY

An aspect of the present invention provides an electrode for measuring a biosignal in which impedance change or the insertion of motion artifact is minimized and which measures a high quality signal therefrom.

According to an aspect of the present invention, there is provided an electrode for measuring a biosignal, the electrode including: a sheet member formed of an insulating material; a conductive member formed of a conductive material on a first surface of the sheet member; and an electrolyte member formed on the sheet member, comprising a gel-type electrolyte having tackiness, and having a base part covering the conductive member and a plurality of protrusions protruding from a surface of the base part which is directed to the skin.

According to an aspect of the present invention, there is provide an electrode, including: an insulating sheet; a conductor on a side of the insulating sheet; and an electrolyte member on the side, the electrolyte member comprising a base covering the conductor and a plurality of protrusions protruding from a side of the base and in a direction away from the conductor, at least the protrusions being comprised of a tacky gel-type electrolyte.

According to another aspect of the present invention, there is provided an electrode, including: an electrolyte portion transmitting a biosignal therethrough, the electrolyte portion having a base and a plurality of protrusions protruding from the base and being attachable to a subject, at least the protrusions comprising a tacky gel-type electrolyte; an insulating sheet; and a conductor between the base and the insulting sheet, the conductor receiving the biosignal from the electrolyte portion.

According to another aspect of the present invention, there is provided a method of measuring a biosignal of a subject, including: attaching an electrode for measuring the biosignal to the subject, the electrode including an electrolyte portion transmitting the biosignal therethrough, the electrolyte portion having a base and a plurality of protrusions protruding from the base and being attachable to the subject, at least the protrusions comprising a tacky gel-type electrolyte, an insulating sheet, and a conductor between the base and the insulting sheet and receiving the biosignal from the electrolyte portion; and receiving the biosignal via the plurality of protrusions.

Additional and/or other aspects and advantages of the present invention will be set forth in part in the description which follows and, in part, will be obvious from the description, or may be learned by practice of the invention.

### BRIEF DESCRIPTION OF THE DRAWINGS

The above and/or other aspects and advantages of the present invention will become apparent and more readily appreciated from the following detailed description, taken in conjunction with the accompanying drawings of which:

FIG. 1 illustrates a conventional electrode for measuring a biosignal in a resting state and a moving state of a body to which the electrode is attached;

FIG. 2 is a perspective view of a plurality of electrodes for measuring a biosignal according to an embodiment of the present invention;

FIG. 3 is a perspective view of a lower portion of one of the electrodes illustrated in FIG. 2;

FIG. 4 is a cross-sectional view of one of the electrodes illustrated in FIG. 2;

FIG. 5 is a perspective view of modification of the electrode for measuring a biosignal illustrated in FIG. 4;

FIG. 6 is an enlarged cross-sectional view of portion of FIG. 4; and

FIG. 7 illustrates the electrode for measuring a biosignal illustrated in FIG. 2 in a resting state and a moving state of a body to which the electrode is attached.

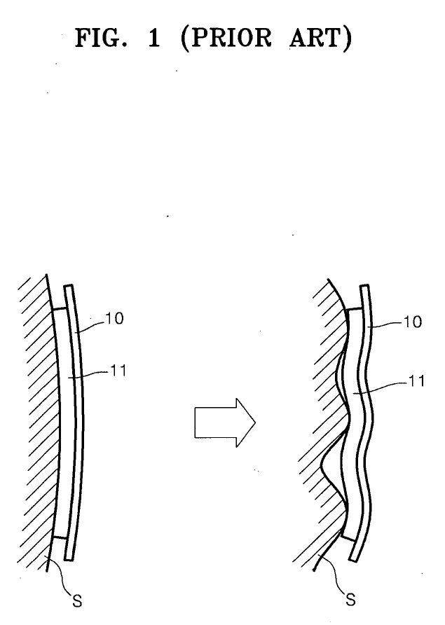

### DETAILED DESCRIPTION OF EMBODIMENTS

Reference will now be made in detail to embodiments of the present invention, examples of which are illustrated in the accompanying drawings, wherein like reference numerals refer to the like elements throughout. The embodiments are described below in order to explain the present invention by referring to the figures.

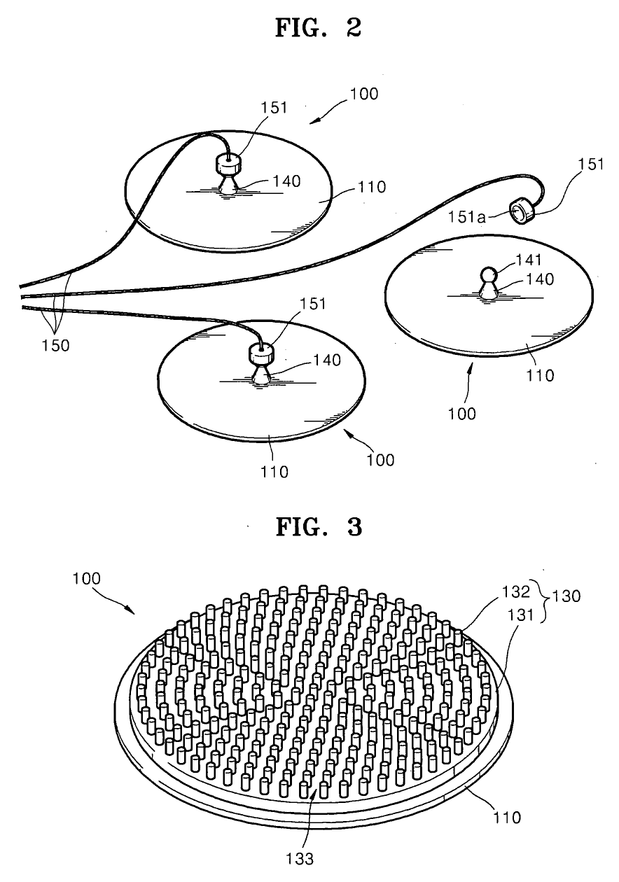

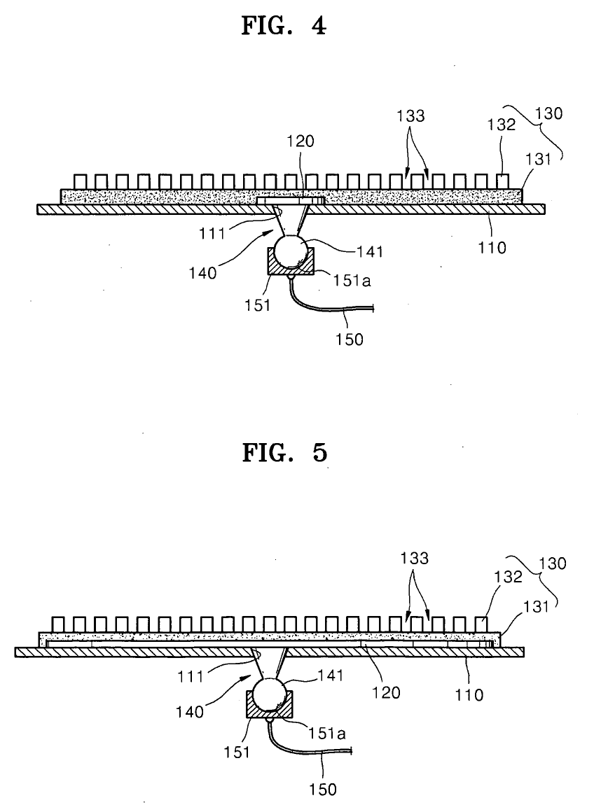

FIG. 2 is a perspective view of a plurality of electrodes for measuring a biosignal according to an embodiment of the present invention, and FIG. 3 is a perspective view of a lower portion of one of the electrodes illustrated in FIG. 2. FIG. 4 is a cross-sectional view of one of the electrodes illustrated in FIG. 2.

Referring to FIG. 2, an electrode 100 for measuring a biosignal can be used, by way of a non-limiting example, to perform an electrocardiograph (ECG) test which is a type of general test performed on the body. In an ECG test, electrical activity generated by the heart at rest and when active is spread into the whole body from the heart and causes an electrical potential difference in two locations of the chest and the electrical potential difference is measured and recorded as a graph.

A plurality of electrodes for measuring a biosignal are disposed so that the electrical potential difference can be measured in at least two locations of the chest. The electrodes 100 for measuring a biosignal are connected to signal analyzers in wired or wireless ways so that signals measured by the electrodes 100 can be interpreted and processed. When the electrodes 100 are connected to the signal analyzers by wires, a snap 140 formed of a conductive material can be disposed on each of the electrodes 100. The snap 140 protrudes from the electrode 100 to be connected to an electric wire 150 drawn from the signal analyzer. The snap 140 is combined with a cap 151 disposed at a terminal of the electric wire 150. A ball 141 is disposed on a terminal from which the snap 140 protrudes so that the snap 140 can rotate freely in the cap 151 when combined with each other. A concave groove 151a is formed in the cap 151 so that more than the half of the ball 141 is inserted into the concave groove 151a, as illustrated in FIG. 4. The electrode 100 for measuring a biosignal can also be used, by way of a non-limiting example, to measure electromyography (EMG).

The electrode 100 for measuring a biosignal will now be described in greater details with reference to FIGS. 3 and 4. Referring to FIGS. 3 and 4, the electrode 100 includes a sheet member 110, a conductive member 120 disposed on one surface of the sheet member 110, and an electrolyte member 130 which is disposed on the sheet member 110 and covers the conductive member 120. The sheet member 110 is formed as a thin plate formed of an insulating material, for example, resin. The sheet member 110 is illustrated as disc shaped, but it is to be understood that other shapes are contemplated.

The conductive member 120 is formed of a conductive material, for example, one element selected from a group of elements consisting of gold (Au), silver (Ag), and copper (Cu). The conductive member 120 is formed on a first surface of the sheet member 110 to a predetermined thickness and may be disposed in a portion of a central area of the sheet member 110. The snap 140, disposed by passing through a hole 111 of the sheet member 110, is combined with a surface of the conductive member 120 which is directed to the sheet member 110. Here, the conductive member 120 and the snap 140 may be formed as a single body. The conductive member 120 detects a biosignal using the electrolyte member 130 that contacts the skin S (shown in FIG. 7) and allows the detected signal to be provided to the signal analyzer via the electric wire 150 that is connected to the snap 140. The conductive member 120 may extend across the first surface area of the sheet member 110, as illustrated in FIG. 5. That is, the conductive member 120 may be disposed across the entire first surface area excluding the edge area of the sheet member 110. As the conductive member 120 is widely disposed across the sheet member 110, the area corresponding to the skin S of the conductive member 120 can be increased. As such, the insertion of motion artifact via the conductive member 120 can be minimized so that a comparatively high quality signal can be obtained.

The conductive member 120 is covered by the electrolyte member 130. The electrolyte member 130 comprises a tacky and electrically conductive gel-type electrolyte. The electrolyte may include water, tartaric acid, glycerine, sodium polyacrylate, and polyethylene glycol. For example, the electrolyte may include 30-70 weight % water, 0.5-5 weight % tartaric acid, 20-50 weight % glycerine, 5-10 weight % sodium polyacrylate, and 1-6 weight % polyethylene glycol.

The water contained in the electrolyte increases electrical conductivity, hydrates and softens stratum corneum to quicken the absorption of sclerite of the electrolyte. Tartaric acid is used to adjust the pH of the electrolyte and to keep the stability of the electrolyte or the absorption state of the electrolyte at sclerite. Glycerine is used for water retention purposes. Sodium polyacrylate is used to maintain the water content and maintain stability and to improve adhesion of sclerite. In addition, sodium polyacrylate is included to prevent the electrolyte from being too sticky, is used to alleviate pain during detachment from the skin and prevents electrolyte components from remaining in the skin. Polyethylene glycol is used to uniformly dissolve electrolyte components or to disperse the electrolyte components, to keep and emit the electrolyte components in the electrolyte stably and to quicken the absorption of sclerite. In addition, polyethylene glycol is used to prevent a conductive material in the electrolyte from being educed as crystal and to quicken the absorption of sclerite.

An outer surface of the electrolyte member 130 formed of the electrolyte contacts the skin S. The electrolyte member 130 may be disposed over the entire first surface of the sheet member 110 so that the area of the electrolyte member 130 that contacts the skin S can be maximized. As illustrated in FIGS. 3 and 4, a portion of the edge of the sheet member 110 may be exposed. The electrolyte member 130 includes a base part 131 formed on the sheet member 110 to a predetermined thickness and a plurality of protrusions 132 that respectively protrude from a first surface of the base part 131 to a predetermined thickness. The overall thickness from the bottom surface of the base part 131 to the top surface of the protrusions 132 is larger than the thickness of the sheet member 110 so that sufficient adhesive force can be obtained between the electrolyte member 130 and the skin S.

The thickness of the base part 131 is larger than the thickness of the conductive member 120 so as to completely cover the conductive member 120. As the protrusions 132 protrude from the base part 131, spaces 133 are formed between the protrusions 132. The spaces 133 serve as paths in which perspiration discharged from the skin S does not remain between the electrolyte member 130 and the skin S but smoothly flows outside when the electrolyte member 130 is attached to the skin S. In addition, air flows in and out freely through the spaces 133 so that the spaces 133 serve to quicken the evaporation of perspiration. The spaces 133 formed by the protrusions 132 are used to minimize perspiration that remain between the electrolyte member 130 and the skin S. Thus, even though the electrolyte member 130 is attached to the skin S for a long time, the tackiness of the electrolyte member 130 is not degraded. As such, the electrolyte member 130 can be attached to the skin S and kept in this state for a long time, and a change of impedance between the electrolyte member 130 and the skin S can be minimized. As a result, a good quality signal transmitted to the conductive member 120 via the electrolyte member 130 can be obtained.

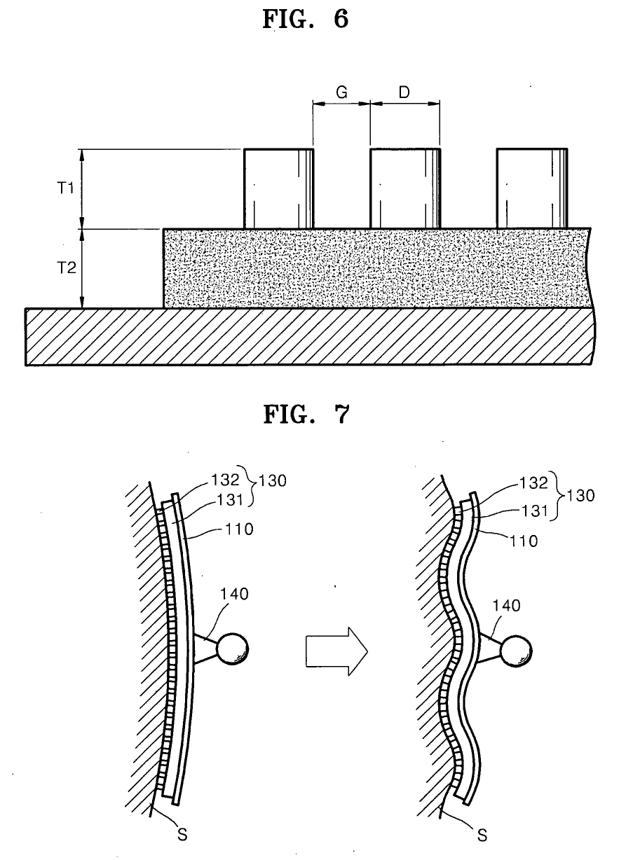

It is to be understood that the protrusions 132 can take the form of various shapes. However, the protrusions 132 are cylindrically shaped according to the present embodiment, and can be arranged at uniform intervals across the entire area of the base part 131, as illustrated in FIG. 3. If the protrusions 132 are cylindrically shaped, outer surfaces of the protrusions 132 can be curved so that the flow of perspiration can be smoothly induced. Since it is advantageous that the spaces 133 between the protrusions 132 are sufficient, a thickness T1 of the protrusions 132 may be the same as or larger than a thickness T2 of the base part 131, as illustrated in FIG. 6. In addition, the larger a distance G between the protrusions 132, the larger the effect of perspiration flowing but the size of a surface in which the protrusions 132 and the skin S contact is reduced so that adhesive force is lowered. Thus, the distance G between the adjacent protrusions 132 may be the same as or smaller than a diameter D of the protrusions 132. Furthermore, the diameter D of the protrusions 132 and the distance G between the adjacent protrusions 132 may be small, for example, several micrometers, so that a sufficient surface in which the protrusions 132 and the skin S contact can be obtained.

As the spaces 133 are formed between the protrusions 132 in this way, the protrusions 132 can move more flexibly. Thus, even in environments where the body performs a motion, for example, when the body bends forward and straightens, the protrusions 132 are closely attached to the skin S and can be maintained in this state. That is, even though the skin S is severely curved along the body as illustrated in the diagram on the right of FIG. 7 when the protrusions 132 are first closely attached to the skin S of the body at rest as illustrated in the diagram on the left of FIG. 7, the protrusions 132 move flexibly along the skin S and are closely attached to the skin S so that the attached state can be maintained. In this manner, even in a motion environment, the electrolyte member 130 can be attached to the skin S. As such, since a change of impedance between the electrolyte member 130 and the skin S can be minimized, the insertion of motion artifact in the signal transmitted to the conductive member 120 via the electrolyte member 130 can be minimized. As a result, a good quality signal can be obtained.

In the electrode for measuring a biosignal according to the above-described embodiments of the present invention, a plurality of protrusions are formed on a surface of an electrolyte member which is directed to the skin, such that perspiration between the electrolyte member and the skin is efficiently removed through spaces disposed between the protrusions. As such, the electrolyte member can be attached to the skin for a long time, and a change of impedance between the electrolyte member and the skin can be minimized. In addition, even in a motion environment, the electrolyte member can be attached to the skin. As such, the insertion of motion artifact in a signal transmitted to a conductive member via the electrolyte member can be minimized such that a good quality signal can be obtained.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. An electrode for measuring a biosignal, comprising:
a sheet member formed of an insulating material;
a conductive member formed of a conductive material on a surface of the sheet member; and
an electrolyte member formed on the sheet member, comprising a tacky gel-type electrolyte, and having a base part covering the conductive member and a plurality of protrusions protruding from a surface of the base part which is directed to skin.

2. The electrode of claim 1, wherein the protrusions are cylindrically shaped and arranged at uniform intervals.

3. The electrode of claim 2, wherein a distance between adjacent protrusions is equal to or less than a diameter of the protrusions.

4. The electrode of claim 2, wherein a thickness of the protrusions is at least equal to a thickness of the base part.

5. The electrode of claim 1, wherein the protrusions are arranged on an entire surface area of the base part.

6. The electrode of claim 5, wherein the protrusions are arranged at uniform intervals across the entire area.

7. The electrode of claim 1, wherein a thickness of the electrolyte member is greater than a thickness of the sheet member.

8. The electrode of claim 1, wherein the electrolyte comprises water, tartaric acid, glycerine, sodium polyacrylate, and polyethylene glycol.

9. The electrode of claim 8, wherein the electrolyte comprises, by weight, 30-70% water, 0.5-5% tartaric acid, 20-50% glycerine, 5-10% sodium polyacrylate, and 1-6% polyethylene glycol.

10. The electrode of claim 1, wherein the conductive material is one of a group consisting of gold (Au), silver (Ag), and copper (Cu).

11. The electrode of claim 1, wherein at least a portion of the conductive member is disposed in a central area of the sheet member.

12. The electrode of claim 11, wherein the electrolyte member is formed so that only a portion of an edge of the sheet member is exposed in the first surface of the sheet member.

13. The electrode of claim 11, wherein the conductive member extends across the first surface of the sheet member from a central area of the sheet member to be near an edge area of the sheet member.

14. The electrode of claim 13, wherein the conductive member is formed of a thin film to have flexibility.

15. The electrode of claim 1, wherein a snap for connection with a signal analyzer is disposed on a second surface of the sheet member.

16. An electrode, comprising:
an insulating sheet;
a conductor on a side of the insulating sheet; and
an electrolyte member on the side, the electrolyte member comprising a base covering the conductor and a plurality of protrusions protruding from a side of the base and in a direction away from the conductor, at least the protrusions being comprised of a tacky gel-type electrolyte.

17. The electrode of claim 16, wherein the sheet member is disc-shaped.

18. The electrode of claim 16, wherein the protrusions are cylindrically-shaped.

19. An electrode, comprising:
an electrolyte portion transmitting a biosignal therethrough, the electrolyte portion having a base and a plurality of protrusions protruding from the base and being attachable to a subject, at least the protrusions comprising a tacky gel-type electrolyte;
an insulating sheet; and
a conductor between the base and the insulting sheet, the conductor receiving the biosignal from the electrolyte portion.

20. A method of measuring a biosignal of a subject, comprising:
attaching an electrode for measuring the biosignal to the subject, the electrode including
an electrolyte portion transmitting the biosignal therethrough, the electrolyte portion having a base and a plurality of protrusions protruding from the base and being attachable to the subject, at least the protrusions comprising a tacky gel-type electrolyte,
an insulating sheet, and
a conductor between the base and the insulting sheet and receiving the biosignal from the electrolyte portion; and
receiving the biosignal via the plurality of protrusions.
